(19) United States
(12) Patent Application Publication
(10) Pub. No.: US 2001/0011084 A1
(43) Pub. Date: Aug. 2, 2001

(54) USE OF ESTROGEN TO MODIFY THE AMOUNT OF SEROTONIN TRANSPORTER OR ITS MRNA

(76) Inventors: GEORGE FINK, EDINBURGH (GB); JUDITH KATHERINE MCQUEEN, EDINBURGH (GB); HELEN WILSON, EDINBURGH (GB)

Correspondence Address: ALLAN RATNER, RATNER & PRESTIA, ONE WESTLAKES BERWYN SUITE 301, PO BOX 980, VALLEY FORGE, PA, 194820980

(21) Appl. No.: 09/194,380

(22) PCT Filed: May 29, 1997
(86) PCT No.: PCT/GB97/01467

§ 371 Date: None

(30) Foreign Application Priority Data
May 29, 1996 (GB) 9611192.7

(51) Int. Cl. A61K 31/56

(52) U.S. Cl. 514/169; 514/170; 514/178; 514/182

(57) ABSTRACT

The present invention relates to the use of oestrogen or a functional equivalent thereof to modify the amount of SERT or of SERT mRNA in order to combat disorders such as depressive disorders, migraine or irritable bowel syndrome. The invention provides medicaments comprising oestrogen or a functional equivalent to be administered in a pharmaceutical format. The invention also provides a method of selecting agents able to act as anti-depressants based on the ability of the agents to affect or mimic the association of SERT and oestrogen.

The present invention relates to the use of oestrogen in affecting the amount of serotonin transporter mRNA, the density of serotonin transporter sites, and to the use of oestrogen to affect mental state and mood, for example to treat depression.

It is known that oestrogen increases the number of 5-HT₂ receptors present in the brain and may therefore be of clinical utility in the treatment of depressive disorders or schizophrenia (see, for example, Fink, in “Serotonin in the Central Nervous System and Periphery”, p 175-187, 1995, Elsevier Science BV, eds Takada and Curzon). However, the mechanism by which oestrogen exerts this effect has not previously been demonstrated.

The most potent anti-depressant drugs are inhibitors of the serotonin transporter (SERT), the molecule responsible for uptake of the serotonin or 5-HT neurotransmitter.

There are two types of inhibitors in clinical use; tricyclic anti-depressants which are phenothiazine derivatives exemplified by imipramine, and, secondly, selective serotonin reuptake inhibitors (SSRIs) exemplified by fluoxetine and paroxetine. The disadvantage of the tricyclic anti-depressants is that they also affect the norepinephrine transporter and several types of neurotransmitter receptors.

It is often assumed, intuitively, that the anti-depressant action of SERT inhibitors is to increase the amount of serotonin at synapses and indeed in whole brain. However, this is not the case. The mode of SSRI action is more complex in that the SSRIs decrease serotonin turnover in brain which may reflect the fact that reuptake of serotonin precedes its conversion to 5-hydroxyindoleacetic acid, a key index of 5-HT turnover (Fuller, in “Neuropharmacology of Serotonin”, p1-20, 1985, Oxford University Press, ed Green). Inhibitors of serotonin reuptake also reduce the firing rate of raphe neurons (Aghajanian et al., in “Psychopharmacology a generation of progress”, p171-183, 1978, Raven Press, New York, ed Lipton et al; Clemens, et al., Endocrinology 100 : 692-698, 1977). Long-term (three weeks) treatment with tricyclic anti-depressants such as desipramine significantly reduced the density of [³H]-imipramine binding sites in rat brain, but [³H]-imipramine binding sites on platelets were also significantly reduced in women with depression who had not received anti-depressants for at least one week before blood sampling (Briley, in “Neuropharmacology of Serotonin”, p50-78, 1985, Oxford University Press, ed Green). These together with data on the interactions between uptake sites, receptor supersensitivity and the activity of serotonin neurons (Gartside, et al., Br. J. Pharmacol 115 : 1054-1070, 1995; Inversen, Biochem Pharmacol, 23 : 1927-1935, 1974) run contrary to the oversimplified view that the anti-depressant action of SSRIs and the less specific tricyclic serotonin reuptake blockers is simply to increase the concentrations of 5-HT at central synapses and in whole brain.

It has now been found that oestrogen has a significant effect on the amount of SERT mRNA content in brain tissue, in particular in the dorsal raphe nucleus (DRN) and is subsequently associated with a significant increase in the SERT binding sites in key areas of the brain. Thus, in contrast to the presumed mode of action of anti-depressant drugs, oestrogen has now been shown to-exert its anti-depressive effects by increasing the amount of SERT sites and SERT gene expression.

This realisation has implications for the understanding of the mechanism of action of SSRIs as well as the role of oestrogen in the control of mood, mental state and behaviour.

The present invention provides an explanation for the anti-depressant action of oestrogen by demonstrating a possible effect on the expression of the SERT gene and/or an effect, which may not involve the gene, but rather the conformation and binding affinity of the SERT. The latter mechanism could involve for example glycosylation and/or phosphorylation sites which are present in the SERT protein (Barker et al, in “Psycho-pharmacology: The Fourth Generation of Progress”, p321-333, 1995, Raven Press, New York, ed Bloom and Kupfer) or other post-transcriptional or post-translational modifications.

In the rat, oestradiol, in its positive feedback mode for luteinizing hormone (LH) release stimulates a massive increase in the expression of 5-HT_{2A} receptor mRNA in the dorsal raphe nucleus (Sumner and Fink, 1993 Mol Cell Neurosci 3 : 83-92), and significant increases in the density of 5-HT_{2A} receptors in several forebrain areas (Sumner and Fink, 1995, J Steroid Biochem Mol Biol 54 : 15-20). The key regulator of serotonergic transmission in brain is the reuptake of extracelluar 5-HT by the 5-HT transporter (SERT) (Amara and Kuhar, 1993, Ann Rev Neurosci 16 : 73-93). We have now investigated the possible effects of oestradiol-17β on the expression of SERT mRNA and the density of SERT binding sites in female rat brain.

Adult female COB Wistar rats were ovariectomized under halothane anaesthesia between 09.00 and 12.00 hours on di-oestrus, and immediately injected s.c. with either 10 μg oestradiol benzoate (OB) in 0.1 ml arachis oil (n=7) or with oil alone (n=7). The rats were killed between 16.30 and 18.00 hours on the following day (time of the oestradiol-induced LH surge) and the brains removed. SERT binding sites were measured by quantitative autoradiography in 20μ coronal cryostat sections, using ³H paroxetine as ligand with 4 μM citalopram to measure non-specific binding (Battaglia et al, 1991, Synapse 8 : 249-260). SERT mRNA levels were determined by in situ hybridization in sections from 8 brains (4 in each group) using a 45 base oligonucleotide probe labelled at the 3′ end with ³⁵S γ-ATP.

The distribution of SERT binding sites in female rat brain was identical to that reported in male brain (de Souza and Kuyatt, 1987, Synapse 1 : 488-496). In OB-treated animals the density of SERT binding sites was significantly higher (Mann-Whitney U test, 2P<0.05) in the basolateral amygdala (20%), lateral septum (90%), ventromedial nucleus of hypothalamus (250%), and ventral nucleus of thalamus (250%) and decreased (by 15%) in periaqueductal central gray. The expression of SERT mRNA was confined to cells of the dorsal and median raphe nuclei. There were significantly more (50%) labelled cells in the dorsal raphe nucleus in sections from OB compared with oil-treated rats.

These results show that oestrogen has potent effects on the serotonin transporter as well as 5-HT_{2A} receptors, suggesting that the effects of oestrogen on mood and mental state may be mediated through both of these central serotonergic mechanisms.

Whilst we do not wish to be bound by theoretical considerations, it is believed that the interaction between oestrogen and SERT is likely to be related to the marked sex difference in the incidence of depression, and to postnatal and perimenopausal depression in particular. It is also believed that oestrogen exerts its effects via the regulatory elements of the SERT gene.

It has not previously been demonstrated that SERT may be the link between the association of oestrogen with depressive disorders, and also with migraine and irritable bowel syndrome. The incidence of migraine is significantly greater in women than in men, as is also the case for depression. The present invention may have relevance to this and the sex difference in schizophrenia.

The present invention may also have relevance to the following conditions in which serotonin has been implicated: affective disorders, anxiety disorders, obsessive-compulsive disorder; schizophrenia; eating disorders; sleep disorders; sexual disorders; impulse disorders; developmental disorders; ageing and neurodegenerative disorders; substance abuse; pain sensitivity; emesis; myoclonus; neuroendocrine regulation; circadian rhythm regulation; stress disorders; carcinoid syndrome.

In one aspect, the present invention provides the use of oestrogen or functional equivalent thereof to modify the amount of SERT or of SERT mRNA in order to combat depressive disorders, migraine or irritable bowel syndrome, or any of the disorders listed above. Generally, the oestrogen or its functional equivalent will be administered in a pharmaceutically acceptable format.

The present invention also provides a method of combatting depressive disorders, migraine, irritable bowel syndrome or any of the disorders listed above in the human or non-human (preferably mammalian) animal body, said method comprising administering to said body a quantity of oestrogen sufficient to increase the amount of SERT.

Viewed from another aspect, the present invention provides a method of combatting depressive disorders, migraine, irritable bowel syndrome or any of the disorders listed above in the human or non-human (preferably mammalian) animal body, said method comprising treating the patient with an agent able to cause an increase in the amount of SERT mRNA, amount or activity of SERT.

The present invention further provides a method of selecting agents able to act as anti-depressants, wherein said agents affect or mimic the association between SERT and oestrogen, or wherein said agents increase the amount of SERT mRNA, of SERT or of the activity of SERT.

The invention will now be further illustrated by reference to the following, non-limiting, examples and the accompanying figures wherein;

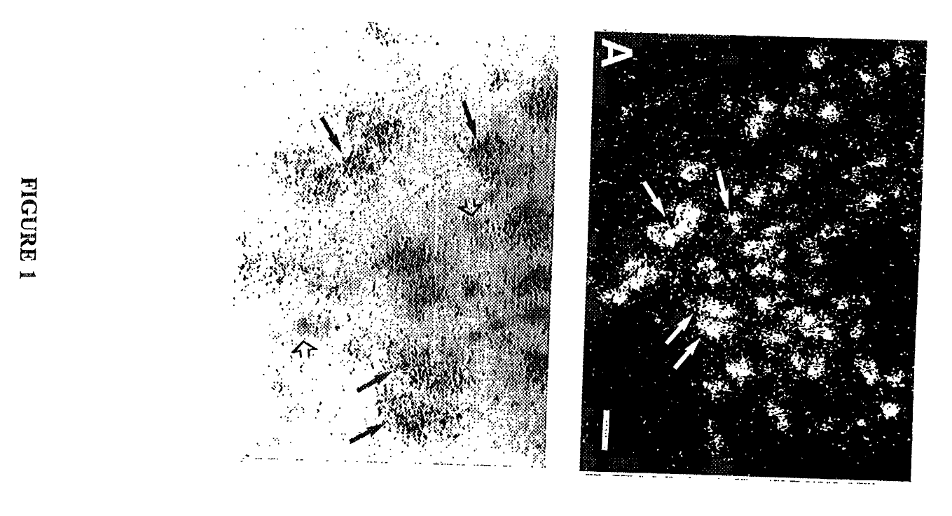

FIG. 1 illustrates Dark-field (A) and higher power bright-field (B) photomicrographs of a coronal section of the ventromedial part of the dorsal raphe nucleus after in situ hybridization with a [³⁵S]-labelled oligonucleotide probe to SERT mRNA. The midline is in the centre of the pictures. The arrows point to the same labelled neurons in A and B. Unlabelled cells are indicated by open arrowheads in B. Scale bar 50 μm.

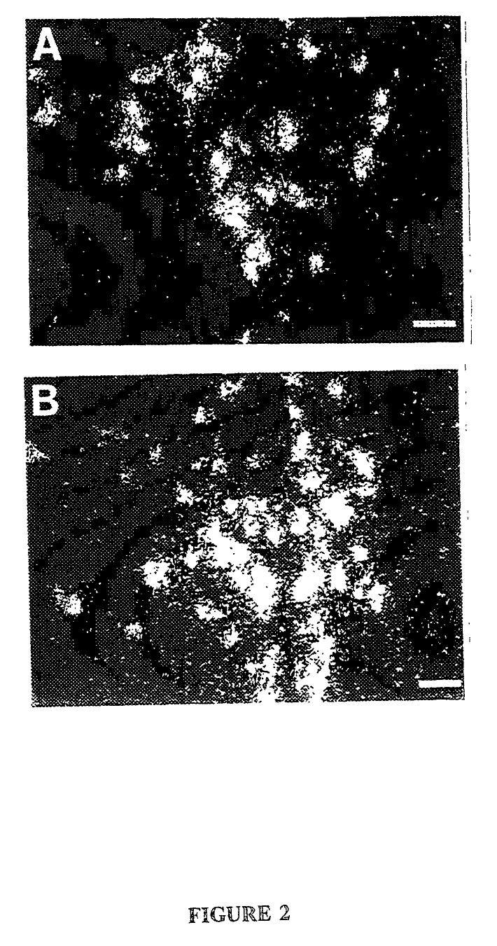

FIG. 2 illustrates Dark-field photomicrographs of the ventromedial part of the dorsal raphe after probing for SERT mRNA. A: control brain, OVX+OIL; B: brain from OVX rat treated with estradiol-17β (EB). Note that after EB treatment (B) there are many more SERT mRNA containing cells than in the control (A). Scale bar 50 μm.

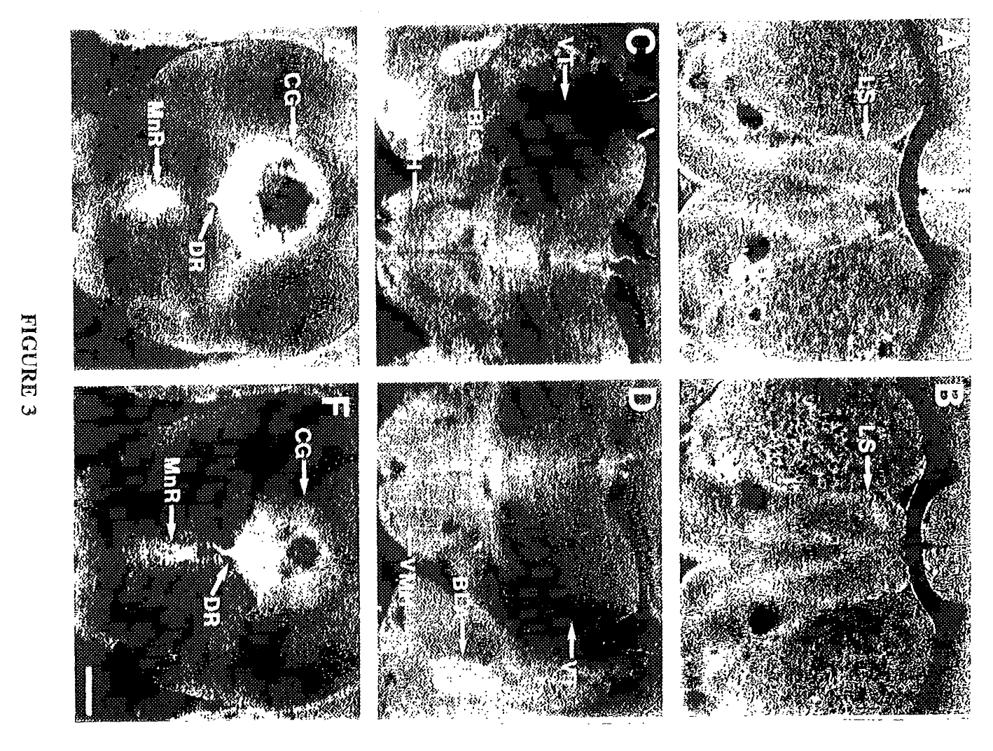

FIG. 3 illustrates Dark-field film autoradiographs showing the regional distribution of [³H]-paroxetine labelled serotonin uptake sites in coronal sections of female rat brain. A, C, E: control brains, OVX+OIL; B, D, F: brains from OVX rats injected with 10 μg estradiol-17β (EB). The density of binding sites in lateral septum (LS), basolateral amygdala (BLA), ventral thalamus (VT) and ventromedial hypothalamic nucleus (VMH) is higher in animals treated with estrogen (B, D) than in controls (A, C). In periaqueductal central gray (CG) the density is lower (F compared to E). There is no apparent difference in labelling in the dorsal raphe (DR) or median raphe (MnR). Scale bar 1 mm.

### EXAMPLE 1

1. Introduction

The spontaneous ovulatory surge of luteinizing hormone (LH) is generated by a positive feedback cascade in which a surge of estradiol-17β (E₂) acts on the brain to trigger a surge of luteinizing hormone releasing hormone (LHRH) [15]. Serotonin (5-HT) plays a central role in the E₂-induced LHRH/LH surge. Recent studies in this laboratory have established that a 5HT_{2A} receptor mechanism is a key component in the E₂-induced LH surge [12], and that E₂ in its positive feedback mode for LH release stimulates a massive increase in the expression of 5-HT_{2A} receptor mRNA in the dorsal raphe nucleus [55] and significantly increases the density of 5-HT_{2A} receptors in frontal, cingulate and primary olfactory cortex, and in the nucleus accumbens [56]. These findings suggest that the 5-HT_{2A} receptor may play a key role in mediating the effects of E₂ on mood and mental state [17, 18, 56].

Serotonergic mechanisms play a pivotal role in depressive illness [27, 34] and depression in women is more common at times of falling estradiol levels [18, 37, 46, 56]. Indeed, estrogen has been shown to be an effective therapy in postnatal [21] and perimenopausal depression [37] and in women with depression resistant to conventional therapy [29]. However, the underlying mechanisms involved are unknown. The key regulator of serotonin neurotransmission in brain is the serotonin transporter (SERT) [3] which rapidly removes serotonin from the synaptic cleft. There is indirect evidence that changes in serotonin uptake may be implicated in depression [34]. Selective serotonin reuptake inhibitors (SSRIs) are potent antidepressant drugs, and we have recently reported a link between the SERT gene and susceptibility to depression [40].

Early attempts to study the effects of steroids on serotonin uptake sites in rat brain were hampered by the use of non-specific ligands and have yielded inconsistent results [reviewed in 35]. The aims of the present study were to determine whether E₂, in its positive feedback mode for stimulating LHRH release, affects SERT mRNA levels and SERT binding sites in brain. SERT mRNA levels were measured by in situ hybridization using a novel oligonucleotide probe derived from the published base sequence for rat SERT mRNA [7]. Changes in SERT binding sites were determined by quantitative autoradiography using the highly selective ligand paroxetine [33].

2. Materials and Methods

2. 1 Animals

Experiments were carried out on adult female COB Wistar rats, 200-250 g body weight, bred in the Department of Pharmacology, University of Edinburgh, and maintained under conditions of controlled lighting (lights on 0500-1900 h) and temperature (22° C.), with free access to food and water. Oestrous cycles were monitored by the daily inspection of vaginal smears and all animals studied had at least 2 consecutive regular cycles. The experimental model was similar to that described by Sumner and Fink (1993) [55]. Briefly, 14 rats were bilaterally ovariectomized (OVX) under general anaesthesia (halothane) on the morning of diestrus between 0900 h and 1200 h, and immediately injected s.c. with either 10 μg estradiol benzoate (EB, Paines and Byrne Limited, West Byfleet, Surrey, UK) in 0.1 ml arachis oil or 0.1 ml arachis oil alone (7 rats per group). This dose of EB produces blood levels of 100-120 pg E₂/ml for up to 30 h in ovariectomized rats [22]. Between 1630 h and 1800 h on the next day (presumptive proestrus) around the expected time of the peak surge of LH, the animals were decapitated and the brains rapidly removed and frozen in isopentane at −48° C. Brains were stored at −70° until sectioning. Examination of the uterine horns in all animals confirmed that those in the EB-treated group were markedly distended with fluid. Plasma r-LH levels, determined in trunk blood by radioimmunoassay using r-LH-RP-2 as reference preparation [12] were 0.9±0.1 ng/ml (mean±sem, n=7) in the oil-injected controls and 7.5±3.1 ng/ml (n=7) in the EB group. This difference is statistically significant (2P<0.01, Wilcoxon Rank Sum Test).

2.2 Preparation of brains

Serial coronal 20 μm sections were cut on a cryostat at −17° C. at the following levels [41]: Area 1 (lateral septum) bregma +0.48 to −0.26 mm; area 2 (hypothalamus) bregma −1.8 to −2.30 mm; area 3 (amygdala) bregma −4.16 to −4.80 mm; area 4 (substantia nigra) bregma −4.80 to −5.30 mm; area 5 (midbrain raphe) bregma −7.30 to −7.80 mm; area 6 (locus coeruleus) bregma −9.30 to −9.80 mm. Thus regions of serotonergic cell bodies (midbrain raphe) and terminals as well as areas important for neuroendocrine function were included. Sections were thaw mounted on to either gelatin plus poly-L-lysine coated slides (for in situ hybridization) or gelatin-subbed slides (for quantitative autoradiography). Slides were stored in sealed plastic boxes at −70° C. until further processing.

2.3 Probe Development

The published nucleotide sequence of the rat SERT mRNA [7] as contained in the EMBL data base (RRSERTRAN rat mRNA for serotonin transporter) was searched for sequences showing poor homology with other transporters and serotonin receptors in rat, mouse and human, but good homology for SERT between species. The program used was the Wisconsin Package, Version 8, August 1994 (Genetics Computer Group, 575 Science Drive, Madison, Wis., USA). A 45-mer oligonucleotide probe complementary to nucleotides 1961-2005 was synthesized by Oswel DNA service, University of Edinburgh, UK. This had a G/C content of 56% and showed 91-93% homology with human and mouse SERT but less than 51% homology with other transporters and serotonin receptors. Thus the risk of cross-hybridization with other receptor and transporter mRNA was minimal. The probe was labelled at the 3′ end with [³⁵S]-dATP (specific activity>1000 Ci/mmol, DuPont (UK) Ltd, Stevenage, Herts, UK). After purification through Nu-Clean D25 spun columns (IBI Ltd, Cambridge, UK), the probe was stored at −70° C. in double strength hybridization buffer without formamide until the next day.

2.4 Prehybridization and Hybridization

Slides through the midbrain raphe in 8 brains (4 oil controls, 4 EB-treated) were thawed at room temperature and fixed in 4% (w/v) paraformaldehyde in 0.1M phosphate buffer, pH 7.5, for 10 min. The slides were washed twice for 5 min in 2×saline sodium citrate (2 ×SSC=0.3M NaCl and 0.03M sodium citrate, pH 7.0) which had 5 drops diethylpyrocarbonate added to each 500 ml 2×SSC just before use. The slides were drained, laid horizontal and covered with 250 μl prehybridization buffer containing: 40% deionized formamide, 0.6M NaCl, 0.01M Tris, pH 7.5, 1 mM EDTA, 0.02% Ficoll, 0.02% polyvinyl-pyrrolidine, 0.1% bovine serum albumin, 0.5 mg/ml sonicated salmon sperm DNA, 0.05 mg/ml glycogen, 0.05 mg/ml yeast t-RNA for 2 h at 37° C. The slides were drained and sections covered with 250 μl of probe (^{˜}1×10⁷ cpm) in hybridization buffer (which was similar to the prehybridization buffer but contained 0.1 mg/ml salmon sperm DNA, 0.005 mg/ml glycogen) and 10% dextran. Just before use, 10 μl 1M dithiothrietol/ml were added. Slides were sealed in a moist chamber and incubated for 20 h at 37° C. After hybridization, the slides were washed at 37° C. for 1 h each in 2×SSC, 1×SSC and 0.5×SSC and then dehydrated for 2 min each in 50%, 70% and 90% ethanol containing 0.3M ammonium acetate. Sections were air-dried overnight at room temperature. Slides were vacuum desiccated for 2 h and then dip-coated in Ilford G5 photographic emulsion (diluted 1:1) and air-dried in total darkness for 18 h. This was followed by exposure, in light tight boxes at 4° C. for 14 days. Emulsion-coated slides were developed in Phenisol for 4 min, fixed in Hypam (2×5 min) and lightly stained with 1% pyronine.

2.5 Controls

In some brains, sections through the midbrain raphe were either hybridized with a 49-mer oligonucleotide probe complementary to the sequence for the glycopeptide domain of rat arginine vasopressin [36] or pretreated with RNAase (800 μg/ml for 1 h at 37° C.) before hybridization with the rat SERT mRNA probe. No positive cells were detected in the midbrain raphe with either treatment.

Sections from the other 5 brain areas in 2 brains were hybridized with the SERT mRNA probe to identify the extent of SERT mRNA labelling throughout the brain.

2.6 Quantitative Autoradiography

Slide-mounted brain sections from one EB-treated and one oil-injected control rat were processed together for [³H]paroxetine autoradiography according to the method of De Souza and Kuyatt (1987) [10] as described by Battaglia et al (1991) [5]. Briefly, the slides were brought to room temperature, incubated for 3 h at room temperature with 250 pM [³H]paroxetine (Specific Activity 18-29 Ci/mmol, DuPont (UK) Limited, Stevenage, Herts, UK) in 50 mM Tris HCl containing 120 mM NaCl and 5 mM KCl (pH 7.7). Non-specific binding was assessed by incubating slides of alternate sections in the presence of 4 μM citalopram (gifted by H Lundbeck, Copenhagen, Denmark). Following incubation, slides were washed (2× 30 min) in buffer at room temperature, dipped in ice-cold distilled water and dried in a vacuum desiccator. The labelled slide-mounted sections and autoradiographic tritiated microscales (Amersham, Little Chalfont, Bucks, UK) were apposed to Hyperfilm (Amersham) and exposed in X-ray cassettes for 8 weeks at −40° C. Each film included matched sections for total and non-specific binding from brains from both treatment groups. Autoradiograms were developed for 4 min in Phenisol (Ilford, UK), and washed and fixed for 10 min in Hypam (Ilford, UK). The slides were stained with pyronine to allow precise neuroanatomical regions to be identified and matched with appropriate regions on the autoradiograms.

2.7 Microscopy and Quantitative Analysis

In Situ Hybridization

Matching sections through the dorsal raphe nucleus at the level of Plate 48 [41] were selected from all 8 brains. Slides were examined under bright-field illumination at ×25 magnification and the total number of labelled cells counted in the dorsal raphe and median raphe nuclei in each of 4 coronal sections. Slides were also analysed by computer-assisted grain counting using the Optomax image analysis system. The area of silver deposit within a cell, and the area of the cell body were measured for 20 cells in the dorsal raphe and 10 cells in the median raphe in each of the 4 matched sections. Grain density was expressed as the percentage of the neuron area occupied by silver deposit. Density measurements were also made over unlabelled cells (8 per brain) in both the dorsal and median raphe nuclei.

[³H]Paroxetine Autoradiography

Selected neuroanatomical regions (19 in total) were identified in the autoradiographs using the stained sections and the atlas of Paxinos and Watson (1986) [41]. Films were analysed for regional optical density using an Optomax image analysis system (Synoptics Ltd, Cambridge, UK) with macroviewer. The minimum area over which density readings could be obtained was 0.05 mm² (for example raphe pontis). Readings for one region were made from at least 4 sections for each brain, and the mean coefficient of variance (SD as % mean) was calculated to be 4.07. A quadratic equation was found to be the best fit for the relationship between optical density and radioactivity of standards included on each film. From the standard curve, optical density readings for individual structures were converted to nCi/mg and then, depending on the specific activity of the [³H]paroxetine used to fmol/mg tissue. Values for specific binding were obtained by subtracting the density of the non-specific binding from the total binding for each neuroanatomical region.

2.8 Statistical Analysis

All comparisons were made using non-parametric statistics (Wilcoxon Rank Sum Test).

3. Results

3.1 In Situ Hybridization of SERT mRNA

High densities of SERT mRNA were found in neurons in the midbrain raphe, particularly in the dorsal and median raphe. Labelled cells appeared as densely packed cell groups within the dorsomedial and ventromedial parts of the dorsal raphe (FIG. 1) with more widely distributed cells in the lateral wings of the nucleus. There was a high signal: background ratio and labelled cells were clearly distinguishable from unlabelled (FIG. 1B). Some neurons in the medial lemniscus were labelled and one or two in the locus coeruleus. However, no other labelled cells were detected in the other brain regions examined. This pattern agrees with the distribution of serotonin-immunoreactive neurons reported by Steinbusch [52]. Low levels of SERT mRNA, as revealed by PCR techniques, have been reported in rat forebrain regions [30], but these were undetected by our methodology.

Table 1 shows that there were about 5 times as many labelled cells in the dorsal raphe than in the median raphe. In the dorsal raphe itself significantly more (2P<0.05, Wilcoxon Rank Sum Test) labelled cells were found in the brains of rats treated with estradiol compared with oil-treated controls (FIG. 2A, 2B and Table 1). Labelled cell counts in the median raphe did not differ significantly between treatment groups.

Image analysis showed that the grain density per cell for labelled cells in both treatment groups and both raphe nuclei were virtually identical (Table 1). There was thus no detectable increase in SERT mRNA expression per cell. The mean size of labelled cells in the median raphe was smaller than in the dorsal raphe but this reached significance (2P<0.01) only when data from both treatment groups were combined. The data for cell area in the dorsal raphe agree with the size for serotonergic neurons [52]. Unlabelled cells in both raphe nuclei were also significantly smaller (2P< 0.01) than labelled cells (Table 1). These cells may represent non-serotonergic neurons. Silver grain deposit over these cells was negligible and constituted <5% that of labelled cells.

3.2 [³H]Paroxetine Quantitative Autoradiography

The pattern of distribution of SERT binding sites in female rat brain was similar to that reported in male brain [5, 10] and is consistent with the organization of serotonergic terminals and cell bodies [52]. High densities of [³H]paroxetine binding were evident in several structures throughout the brain, in particular in the raphe complex and parts of the hippocampus, thalamus and limbic system. FIG. 3 shows representative autoradiograms at selected levels and Table 2 shows a summary of the values for specific binding in 19 anatomical regions given in rostro-caudal order. There were significant differences (Wilcoxon Rank Sum Test) between the 2 treatment groups in 5 of the 19 neuroanatomical regions analysed. Density of binding sites was significantly increased following estradiol treatment in lateral septum (FIG. 3B compared to FIG. 3A); basolateral amygdala and ventromedial nucleus of hypothalamus (FIG. 3D compared to FIG. 3C); and ventral thalamus, which includes posteromedial, posterolateral and ventrolateral thalamic nuclei, in which the levels of specific binding were very low in oil-treated animals. Density of binding sites was significantly decreased in periaqueductal central gray (FIG. 3F compared to FIG. 3E). Although levels tended to be lower in regions of the raphe complex (Table 2) this was not statistically significant. We found no evidence of a change in [³H]paroxetine binding in cingulate and frontal cortex, areas which show dramatic alterations in 5-HT_{2A} receptor binding after estradiol [56].

4. Discussion

The key findings of this study are that estradiol-17β, in its positive feedback mode for LHRH and LH release, increases by about 50% the number of cells in the dorsal raphe nucleus that express SERT mRNA, and the density of paroxetine-labelled serotonin binding sites in lateral septum (90%), basolateral amygdala (20%), ventromedial nucleus of hypothalamus (250%) and ventral nucleus of the thalamus (250%). Estradiol decreases by 15% the number of binding sites in periaqueductal central gray.

4.1 Changes in SERT mRNA Levels SERT mRNA was localized almost exclusively in neurons in the dorsal and median raphe nuclei. The few labelled cells in medial lemniscus and locus coeruleus presumably represent serotonergic cells reported in these regions [52]. While low expression of SERT mRNA in other areas of brain, for example 5-HT terminal areas, cannot be excluded, we could not detect any with our methodology. This distribution of SERT mRNA is consistent with its presence within serotonergic cell bodies. The dendrites of these neurons also carry SERT binding sites which are involved in fine control of serotonergic cell firing regulated through a 5-HT_{1A} inhibitory autoceptor [25].

The increased levels of SERT mRNA (as reflected by the number of labelled cells) in the dorsal raphe was not translated into increased density of paroxetine binding sites within the raphe itself, but binding sites were significantly increased in some terminal areas. The fact that the number of SERT mRNA-containing cells, but not the grain density per cell, was increased could be due to experimental conditions failing to differentiate degree of labelling. However, there are other examples of “all-or-none” effects on mRNA levels, for example the effects of testosterone on AVP expression in the bed nucleus of the stria terminalis [49]. Also, in the prepubertal female rat the al adrenergic antagonist prazosin reduces the total number of LHRH mRNA containing cells without affecting LHRH mRNA concentration per cell [48].

There were no significant differences between treatment groups in labelled cell number in the median raphe. Median raphe serotonergic neurons are reported to inhibit LH release by a GABAergic mechanism [38] while serotonergic neurons in the dorsal raphe stimulate LH release by an adrenergic mechanism involving the locus coeruleus [39]. The differential effect of E₂ on the dorsal compared with the median raphe may be due to the apparent absence of estrogen receptors in the latter [43].

The mechanisms by which SERT gene expression is regulated remain to be elucidated. In rats, chronic administration of SSRIs reduces SERT mRNA concentrations in raphe homogenates [30]. In the same study, 5-HT receptor agonists had no effect on SERT mRNA levels suggesting that serotonin does not regulate its transporter indirectly by a 5-HT_{1A}, 5-HT_{1C} or 5-HT₂ receptor. Antidepressant drugs may exert a direct effect on SERT gene transcription analogous to their effects on type II glucocorticoid receptor gene expression [42]. Further studies will be needed to establish whether the E₂ effects on SERT mRNA levels involve changes in gene transcription or mRNA stability.

4.2 Changes in Paroxetine-Labelled SERT Binding Sites

The distribution of SERT binding sites in the ovariectomized female rat brain appeared similar to that in male rats [5] with the highest levels in the midbrain raphe complex. There were significant differences in the density of binding sites between the EB-treated and control groups in 5 of the 19 brain regions analysed. Significant increases were found in lateral septum, basolateral amygdala, ventromedial nucleus of hypothalamus and ventral nuclei of thalamus, an area with very low levels in control animals. We detected no changes in SERT binding sites in those areas of cortex in which increases in post-synaptic 5-HT2A receptors had previously been reported [56]. This is in agreement with the findings of Mendelson et al (1993) [35] that chronic (7 days) EB does not affect paroxetine binding in cingulate and temporal-parietal cortex.

In only one brain region, periaqueductal central gray, was the density of binding sites significantly reduced in EB-treated rats. This area is important for lordosis behaviour in the rat [50]. Serotonergic innervation from the dorsal raphe exerts an inhibitory influence on lordosis behaviour [47], which is regulated primarily by progesterone, possibly through a non-genomic action [14]. The changes in SERT binding sites in central gray may reflect E₂-induced changes in the activity of this pathway in relationship to its role in lordosis behavior.

Within the raphe, paroxetine-labelled uptake sites, thought to be on the dendrites of the serotonin neurons, do not appear to be as sensitive to change as those in the terminal areas such as cortex and hippocampus. The neurotoxin methylenedioxy-amphetamine (MDA) reduces paroxetine-labelled 5-HT uptake sites by 70% in several brain regions, but the density of binding in the raphe nuclei is unaffected [28]. Similarly, imipramine binding in raphe is unaltered by parachloro-amphetamine which depletes serotonin [23].

The brain regions in which EB induced significant changes in SERT binding all contain high concentrations of estrogen receptors [43]. Steroids can exert either inhibitory or stimulatory effects; in neuroendocrine systems the former are rapidly acting (min) while the latter have a long latency (hours to days) [16]. The classical genomic action of steroids requires activation of steroid receptors but extragenomic membrane effects are also possible [32]. Therefore, the fact that all the regions showing changes in SERT binding sites contain estrogen receptors does not necessarily indicate that E₂ is acting exclusively by a direct and/or genomic action at these sites.

Previous studies of the effects of gonadal steroids on 5-HT uptake sites have focused on cortex and hippocampus, with contradictory results. For example, chronic EB increases imipramine binding in cortex in male rats [45] while paroxetine-labelled uptake sites in cortex are unaffected [35]. Gonadectomy increases imipramine binding in hippocampus in male rats [51] and paroxetine labelling of hippocampus in female and male rats is decreased by chronic EB treatment [35]. Studies using imipramine are confounded by the fact that it labels noradrenaline as well as serotonin reuptake and postsynaptic sites [54]. There have been no previous detailed studies on the short-term (28-30 h) effects of estrogen on paroxetine binding in hypothalamic and limbic areas.

4.3 Circuitry Involved

There are 3 major ascending efferent fibre systems from the midbrain raphe serotonergic neurons [53] and overlapping topographical distribution of dorsal and median raphe efferents in forebrain areas [23]. The pathway of most relevance to this study is the ventral ascending or mesolimbic pathway as it innervates all the regions which showed significant increases in paroxetine binding sites. No changes were shown in caudate nucleus (innervated by mesostriatal pathway) or in substantia nigra (medial ascending pathway). It is possible that E₂ preferentially activates one serotonergic pathway. Certainly the area of the dorsal raphe which showed a significant increase in SERT mRNA-containing cells after EB treatment is also the origin of the mesolimbic pathway.

4.4 Functional Significance

The areas showing changes in SERT binding sites, in lateral septum, amygdala and hypothalamus are integrated components of the limbic and hypothalamic systems, which, through extensive and reciprocal interconnections with limbic telencephalic and diencephalic areas are involved in a variety of physiological behavioural and emotional processes related to higher cognitive as well as neuroendocrine functions. With respect to the latter, the present findings suggest that the SERT may play a key role in the serotonergic mechanism that mediates induction of the LHRH/LH surge [15].

The E₂-induced changes in SERT binding sites could, by altering the function of the brain regions mentioned above, result in significant changes in mental state, mood, emotion and/or behavior. Thus, for example, the amygdala plays a pivotal role in emotion, memory, reproductive and aggressive behavior and neuroendocrine control [2, 6]. The basolateral amygdala, in the rat, has been shown to be involved together with the ventral striatum in stimulus-reward mechanisms [13]. The lateral septum, through its reciprocal connections with the periventricular hypothalamus, plays a key role in neuroendocrine control, and through its connections with the lateral hypothalamus is involved with the control of water and salt intake and thermoregulation [26]. The lateral septum is also implicated in aggression, socially and sexually related behaviours and integrated behaviours such as the relief of fear [26]. The lateral septum receives a dense innervation of vasopressinergic neurons which have their cell bodies in the bed nucleus of the stria terminalis (BNST). Sensitive to control by estrogen and testosterone, this BNST-lateral septal vasopressinergic system is involved in ‘social/olfactory’ memory [11, 16, 26, 49] which could conceivably also be affected by estrogen-induced changes in SERT sites.

The low concentration of SERT sites in the ventral thalamic nuclei requires cautious interpretation of the 250% increase in the density of SERT sites in OB-treated animals. However, these are the major relay nuclei in the reciprocal connections between the deep nuclei of the cerebellum, the somatosensory cerebral cortex and the basal ganglia [44] and, conceivably, relatively massive, estrogen-induced changes in the density of even a small number of SERT sites could have an important modulatory control on impulse traffic in relation to the sensory-motor function of the thalamus.

Taken together with the earlier findings of alterations in 5-HT_{2A} receptors in the same neuroendocrine model [55, 56], our results on SERT mRNA levels and SERT binding sites suggest that effects of estrogen on mood and mental state may be mediated through both of these central serotonergic mechanisms. The action of E₂ on SERT may be a factor in the major sex difference in the incidence of depression, and the possible role of E₂ in postnatal and perimenopausal depression as well as the depressive symptoms of the premenstrual syndrome.

Although SERT inhibitors are potent anti-depressants, the role of the SERT in affective disorders is not clear. Thus, contrary to intuition, SSRIs do not increase brain serotonin levels [e.g. refs 1 and 31]. Rather, SERT inhibitors decrease serotonin turnover in brain, which may reflect the fact that reuptake of serotonin precedes its conversion to 5-hydroxyindoleacetic acid (5-HIAA) [19], and reduce the firing rate of raphe neurons [1, 9]. Long-term (three weeks) treatment with tricyclic antidepressants such as desipramine did significantly reduce the density of [³H]-imipramine binding sites in rat brain, but [³H]-imipramine binding sites on platelets were also significantly reduced in women with depression who had not received antidepressants for at least one week before blood sampling [8]. These together with data on the interactions between uptake sites, receptor supersensitivity and the activity of serotonin neurons [20, 24] run against the oversimplified view that the antidepressant action of SSRIs and the less specific tricyclic reuptake blockers is simply to increase the concentrations of 5-HT at central synapses or in whole brain.

Our present findings, while not providing an answer to some of the paradoxical data outlined above, provide the platform for analysing the way in which a surge of estrogen affects the SERT gene and the density of SERT sites in brain. Our data suggest that the two effects may be distinct. With respect to the effect of estrogen on the SERT gene, it is relevant that the SERT gene possesses an AP-1 site in the second intron, close to a variable-number-tandem-repeat region which we have shown is linked with susceptibility to depression [40]. Secondly, with respect to a possible nongenomic effect on the SERT, the SERT protein has several glycosylation and phosphorylation sites [41] which provide the opportunity for powerful post-translational modification of the affinity of the SERT for 5-HT and SSRIs such as paroxetine. Identification of the site and action of estrogen involved in its effects on central serotonergic mechanisms is the subject of further studies.

TABLE 1
Effects of acute estradiol in ovariectomized rats on SERT mRNA expression
in dorsal and median raphe nuclei. Results as means ± sem.

| | DORSAL RAPHE | | MEDIAN RAPHE | |
|---|---|---|---|---|
| | OVX + OIL | OVX + EB | OVX + OIL | OVX + EB |
| | n = 4 | n = 4 | n = 4 | n = 4 |
| Labeled cells | | | | |
| Number/section | 106.8 ± 8.0 | 158.1 ± 17.8* | 21.6 ± 2.9 | 28.7 ± 3.6 |
| Cell area (μ²) | 280 ± 23 | 287 ± 34 | 225 ± 5 | 230 ± 33 |
| Grain density (% cell area) | 16.0 ± 0.85 | 16.4 ± 1.19 | 16.0 ± 0.63 | 16.1 ± 0.46 |
| Total no. of cells analysed | 320 | 320 | 158 | 160 |
| Unlabeled cells | | | | |
| Cell area (μ²) | 162 ± 8 | 160 ± 35 | 134 ± 31 | 132 ± 22 |
| Grain density (% cell area) | 0.62 ± 0.07 | 0.57 ± 0.13 | 0.55 ± 0.09 | 0.58 ± 0.07 |
| Total no. of cells analysed | 32 | 32 | 32 | 32 |

OVX + OIL, ovariectomized rat treated with oil;
OVX + EB, ovariectomized rat given estradiol benzoate (10 μg s.c.)
*Significantly different from OVX + OIL, 2P < 0.05, Wilcoxon Rank Sum

The number of labeled (SERT mRNA containing) cells was counted in the dorsal and median raphe nuclei in 4 sections at the level of Plate 48 (7.64 mm caudal to bregma) in Paxinos and Watson [41]. The mean value per section was calculated for each brain and a mean value computed for each treatment group in the Table. The total numbers of labeled cells counted in any one brain ranged from 375 to 816 in dorsal raphe and 66 to 146 in median raphe.

TABLE 2
Effects of acute estradiol in ovariectomized rats on [³H]-paroxetine labeled
serotonin reuptake sites in different brain regions. Results of specific
binding as fmol/mg tissue, mean ± sem (number of brains).

| Brain region | OVX ± OIL | OVX + EB |
|---|---|---|
| Frontal cortex | 13.1 ± 3.0 (7) | 21.4 ± 2.4 (7) |
| Cingulate cortex | 47.3 ± 8.1 (6) | 50.9 ± 4.9 (7) |
| Lateral septum | 21.4 ± 4.4 (7) | 40.9 ± 5.5 (7)*↑ |
| Basolateral amygdala | 94.3 ± 6.4 (7) | 112.7 ± 9.2 (7)*↑ |
| Thalamus: Dorsal | 79.7 ± 11.7 (7) | 103.1 ± 11.6 (7) |
| Ventral | 6.0 ± 3.4 (7) | 23.2 ± 5.4 (5)*↑ |
| Paraventricular | 112.7 ± 15.3 (7) | 127.5 ± 8.6 (7) |
| Hypothalamus: Ventromedial | 13.9 ± 4.6 (7) | 51.0 ± 8.3 (6)**↑ |
| Hippocampus: CA1 | 49.3 ± 4.8 (3) | 41.3 ± 7.8 (3) |
| CA3 | 72.1 ± 9.6 (6) | 85.3 ± 5.2 (7) |
| Lateral geniculate | 87.6 ± 14.4 (7) | 92.3 ± 6.2 (7) |
| Superior colliculus | 110.9 ± 10.4 (7) | 110.0 ± 6.7 (7) |
| Periaqueductal central gray | 92.4 ± 4.0 (7) | 76.1 ± 5.5 (7)*↓ |
| Rostral linear nucleus | 109.0 ± 13.7 (5) | 92.7 ± 13.5 (7) |
| Dorsal raphe | 226.0 ± 47.2 (7) | 206.6 ± 10.1 (7) |
| Median raphe | 148.3 ± 18.1 (7) | 146.6 ± 10.3 (7) |
| Raphe pontis | 151.3 ± 44.1 (5) | 124.3 ± 9.1 (4) |
| Locus coeruleus | 206.1 ± 86.5 (7) | 173.0 ± 20.4 (7) |
| Dorsal tegmental nucleus | 155.0 ± 35.1 (5) | 165.2 ± 40.8 (5) |

Note: OVX + OIL, ovariectomized rat treated with oil;
OVX + EB, ovariectomized rat given cestradiol benzoate (10 μg s.c.)
*2P < 0.05; **2P < 0.01 Wilcoxon Rank Sum Test
↑, significant increase; ↓ significant decrease

### REFERENCES

[1] Aghajanian, G. K. and Wang, R. Y., Physiology and pharmacology of central serotonergic neurons. In M. A. Lipton, J. A. DiMascio and K. F. Killam (Eds.), Psychopharmacology: a generation of progress, Raven Press, New York, 1978, pp. 171-183.

[2] Alheid, G. F., de Olmos, J. S. and Beltramino, C. A., Amygdala and extended amygdala. In G. Paxinos (Eds.), The Rat Nervous System, Academic Press, San Diego, 1995, pp. 495-578.

[3] Amara, S. G. and Kuhar, M. J., Neurotransmitter transporters: recent progress, Annu. Rev. Neurosci., 16 (1993) 73-93.

[4] Barker, E. L. and Blakely, R. D., Norepinephrine and serotonin transporters. In F. E. Bloom and D. J. Kupfer (Eds.), Psychopharmacology: The Fourth Generation of Progress, Raven Press, New York, 1995, pp. 321-333.

[5] Battaglia, G., Sharkey, J., Kuhar, M. J. and De Souza, E. B., Neuroanatomic specificity and time course of alterations in rat brain serotonergic pathways induced by MDMA (3,4 methylenedioxymethamphetamine): assessment using quantitative autoradiography, Synapse, 8 (1991) 249-260.

[6] Ben Ari, Y., The Amygdaloid Complex. Elsevier/North Holland Biomedical Press, Amsterdam, 1981, pp. 516.

[7] Blakely, R. D., Berson, H. E., Fremean, R. T., Caron, M. G., Peek, M. M., Prince, H. K. and Bradley, C. C., Cloning and expression of a functional transporter from rat brain, Nature, 354 (1991 ) 66-70.

[8] Briley, M., Imipramine binding: its relationship with serotonin uptake and depression. In R. A. Green (Ed.), Neuropharmacology of Serotonin, Oxford University Press, Oxford, 1985, pp. 50-78.

[9] Clemens, J. A., Sawyer, B. D. and Cerimele, B., Further evidence that serotonin is a neurotransmitter involved in the control of prolactin secretion, Endocrinology, 100 (1977) 692-698.

[10] De Souza, E. B. and Kuyatt, B. L., Autoradiographic localization of ³H-paroxetine-labelled serotonin uptake sites in rat brain, Synapse, 1(1987)488-496.

[11] De Vries, G. J., Sex differences in neurotransmitter systems, J. Neuroendocrinol., 2(1990) 1-13.

[12] Dow, R. C., Williams, B. C., Bennie, J., Carroll, S. and Fink, G., A central 5-HT₂ receptor mechanism plays a key role in the proestrous surge of luteinizing hormone in the rat, Psychoneuroendocrinology, 19 (1994) 395-399.

[13] Everitt, B. J., Morris, K. A., O'Brien, A. and Robbins, T. W., The basolateral amygdala-ventral striatal system and conditioned place preference: further evidence of limbic-striatal interactions underlying reward-related processes, Neuroscience, 42 (1991) 1-18.

[14] Farmer, C. J., Isakson, T. R., Coy, D. J. and Renner, K. J., In vivo evidence for progesterone dependent decreases in serotonin release in the hypothalamus and midbrain central grey: relation to the induction of lordosis, Brain Res., 711 (1996) 84-92.

[15] Fink, G., The GW Harris lecture: “Steroid control of brain and pituitary function”, Q. J. Exp. Physiol., 73 (1988) 257-293.

[16] Fink, G., Molecular principles from neuroendocrine models: steroid control of central neurotransmission, Prog. Brain Res., 100 (1994) 139-147.

[17] Fink, G., The psychoprotective action of estrogen is mediated by central serotonergic as well as dopaminergic mechanisms. In A. Takada and G. Curzon (Eds.), Serotonin in the Central Nervous System and Periphery, Elsevier Science BV, 1995, pp. 175-182.

[18] Fink, G., Sumner, B. E. H., Rosie, R., Grace, O. and Quinn, J. P., Estrogen control of central neurotransmission: effect on mood, mental state and memory, Cell. Mol. Neurobiol., 16 (1996) 325-344.

[19] Fuller, R. W., Drugs altering serotonin synthesis and metabolism. In R. A. Green (Ed.), Neurophaxmacology of Serotonin, Oxford University Press, Oxford, 1985, pp. 1-20.

[20] Gartside, S. E., Umbers, V., Hajos, M. and Sharp, T., Interaction between a selective 5-HTA receptor antagonist and an SSRI in vivo: effects on 5-HT cell firing and extracelluar 5-HT, Br. J. Pharmacol., 115 (1995) 1054-1070.

[21] Gregoire, A. J. P., Kumar, R., Everitt, B., Henderson, A. F. and Studd, J. W. W., Transdermal oestrogen for treatment of severe postnatal depression, Lancet, 347 (1996) 930-933.

[22] Henderson, S. R., Baker, C. and Fink, G., Oestradiol-17μ and pituitary responsiveness to luteinizing hormone releasing factor in the rat: a study using rectangular pulses of oestradiol-17μ monitored by non-chromatographic radioimmunoassay, J. Endocrinol., 73 (1977) 441-453.

[23] Hensler, J. G., Ferry, R. C., Labow, D. M., Kovachich, G. B. and Frazer, A., Quantitative autoradiography of the serotonin transporter to assess the distribution of serotonergic projections from the dorsal raphe nucleus, Synapse, 17 (1994) 1-15.

[24] Iversen, L. L., Uptake mechanisms for neurotransmitter amines, Biochem. Phazmacol., 23 (1974)1927-1935.

[25] Jacobs, B. L. and Fornal, C. A., Activity of brain serotonergic neurons in the behaving animal, Pharmacol. Rev., 43 (1991) 563-578.

[26] Jakab, R. L. and Leranth, C., Septum. In G. Paxinos (Eds.), The Rat Nervous System, Academic Press, San Diego, 1995, pp. 495-578.

[27] Kaplan, H. I. and Sadock, B. J., Comprehensive Textbook of Psychiatry. Williams and Wilkins, Baltimore, Md., 1985.

[28] Kelly, P. A. T., Ritchie, I. M., McBean, D. E., Sharkey, J. and Olverman, H. J., Enhanced cerebrovascular responsiveness to hypercapnia following depletion of central serotonergic terminals, J. Cere. Blood Flow Metab., 15 (1995) 706-713.

[29] Klaiber, E. L., Broverman, D. M., Vogel, W. and Kobayashi, Y., Estrogen therapy for severe persistent depressions in women, Arch. Gen. Psychiatry, 36 (1979) 550-554.

[30] Lesch, K. P., Aulakh, C. S., Wolozin, B. L., Tolliver, T. J., Hill, J. L. and Murphy, D. L., Regional brain expression of serotonin transporter mRNA and its regulation by reuptake inhibiting antidepressants, Mol. Brain Res., 17 (1993) 31-35.

[31] Maes, M. and Meltzer, H. Y., The serotonin hypothesis of major depression. In F. E. Bloom and D. J. Kupfer (Eds.), Psychopharmacology: The Fourth Generation of Progress, Raven Press, New York, 1995, pp. 933-944.

[32] McEwen, B. S., Non-genomic and genomic effects of steroids on neural activity, Trends Pharmacol. Sci., 12 (1991) 141-147.

[33] Mellerup, E. T. and Plenge, P., High affinity binding of ³H-paroxetine and ³H-imipramine to rat neuronal membranes, Psychopharmacology, 89 (1986) 436-439.

[34] Meltzer, H. Y., Role of serotonin in depression, Ann. N.Y. Acad. Sci., 600 (1990) 486-500.

[35] Mendelson, S. D., McKittrick, C. R. and McEwen, B. S., Autoradiographic analyses of the effects of estradiol benzoate on [³H] paroxetine binding in the cerebral cortex and dorsal hippocampus of gonadectomized male and female rats, Brain Res., 601 (1993) 299-302.

[36] Miller, M. A., Urban, J. H. and Dorsa, D. M., Steroid dependency of vasopressin neurons in the bed nucleus of the stria terminalis by in situ hybridization, Endocrinology, 125 (1989) 2335-2340.

[37] Montgomery, J. C., Brincat, M. and Tapp, A., et al, Effect of oestrogen and testosterone implants on psychological disorders in the climacteric, Lancet, i (1987) 297-299.

[38] Morello, H., Caligaris, L., Haymal, B. and Taleisnik, S., Inhibition of proestrous LH surge and ovulation in rats evoked by stimulation of the medial raphe nucleus involves a GABA-mediated mechanism, Neuroendocrinology, 50 (1989) 81-87.

[39] Morello, H. and Taleisnik, S., The inhibition of proestrous LH surge and ovulation in rats bearing lesions of the dorsal raphe nucleus is mediated by the locus coenuleus, Brain Res., 440 (1988) 227-231.

[40] Ogilvie, A. D., Battersby, S., Bubb, V. J., Fink, G., Harmar, A. J., Goodwin, G. M. and Smith, C. A. D., Polymorphism in serotonin gene associated with susceptibility to major depression, Lancet i (1996) 731 -733.

[41] Paxinos, G. and Watson, C., The Rat Brain in Stereotaxic Coordinates. 2nd Edition. Academic Press, Sydney, 1986.

[42] Pepin, M. -C., Govindan, M. V. and Barden, N., Increased glucocorticoid receptor gene promoter activity after antidepressant treatment, Mol. Pharmacol., 41 (1992) 1016-1022.

[43] Pfaff, D. and Keiner, M., Atlas of estradiol-concentrating cells in the central nervous system of the female rat, J. Comp. Neurol., 151 (1973) 121-158.

[44] Price, J. L., Thalamus. In G. Paxinos (Eds.), The Rat Nervous System, Academic Press, San Diego, 1995, pp. 495-578.

[45] Ravizza, L., Nicoletti, F., Pozzi, O. and Barbaccia, M. L., Repeated daily treatments with estradiol benzoate increase the [³H] imipramine binding in male rat frontal cortex, Eur. J. Pharmacol., 107 (1985) 395-396.

[46] Reid, R. L. and Yen, S. S. C., Premenstrual syndrome, Am. J. Obstet Gynecol., 139 (1981) 85-104.

[47] Renner, K. J., Krey, L. C. and Luine, V. N., Effect of progesterone on monoamine turnover in the brain of the estrogen-primed rat, Brain Res. Bull., 19 (1987) 195-202.

[48] Rosie, R., Sumner, B. E. H. and Fink, G., An α₁ adrenergic mechanism mediates estradiol stimulation of LHRH mRNA synthesis and estradiol inhibition of POMC mRNA synthesis in the hypothalamus of the prepubertal female rat, J. Steroid Biochem. Mol. Biol., 49 (1994) 399-406.

[49] Rosie, R., Wilson, H. and Fink, G., Testosterone induces an all-or-none exponential increase in arginine vasopressin mRNA in the bed nucleus of stria terminalis of the hypogonadal mouse, Mol. Cell. Neurosci., 4 (1993) 121-126.

[50] Sakuma, Y. and Pfaff, W. D., Mesencephalic mechanisms for integration of female reproductive behavior in the rat, Am. J. Physiol., 237 (1979) R285-R290.

[51] Sandrini, M., Vergoni, A. V. and Bertoli, A., [³H]Imipramine binding in discrete brain areas is affected by castration in male rats, Brain Res., 496 (1989) 29-34.

[52] Steinbusch, H. W. M., Distribution of serotonin-immunoreactivity in the central nervous system of the rat-cell bodies and terminals, Neuroscience, 6 (1981 ) 557-618.

[53] Steinbusch, H. W. M. and Nieuwenhuys, R., The raphe nuclei of the rat brainstem: A cytoarchitectonic and immunohistochemical study. In P. C. Emson (Eds.), Chemical Neuroanatomy, Raven Press, New York, 1983, pp. 131-207.

[54] Stockert, M., Veiga, A., Butman, S. and De Robertis, E., Pre- and postsynaptic localization of ³H-imipramine binding sites in rat cerebral cortex, J. Recept. Res., 4 (1984) 755-771.

[55] Sumner, B. E. H. and Fink, G., Effects of acute estradiol on 5-hydroxytryptamine and dopamine receptor subtype mRNA expression in female rat brain, Mol. Cell. Neurosci., 4 (1993) 83-92.

[56] Sumner, B. E. H. and Fink, G., Estrogen increases the density of 5-hydroxytryptamine₂A receptors in cerebral cortex and nucleus accumbens in the female rat, J. Steroid Biochem. Mol. Biol., 54 (1995) 15-20.

1. The use of oestrogen or a functional equivalent thereof to modify the amount of SERT or of SERT mRNA in an individual.

2. Use of oestrogen or a functional equivalent thereof in the preparation of a medicament to modify the amount of SERT or of SERT mRNA in an individual.

3. Use of oestrogen or a function equivalent thereof in the preparation of a medicament as claimed in claim 2 to combat a disorder chosen from the group of disorders including affective disorders, anxiety disorders, obsessive-compulsive disorder; schizophrenia; eating disorders; sleeping disorders; sexual disorders; impulse disorders; developmental disorders; ageing and neurodegenerative disorders; substance abuse; pain sensitivity; emesis; myoclonus; neuroendocrine regulation; circadian rhythm regulation; stress disorders; carcinoid syndrome; depressive disorders (but excluding postnatal depression and treatment-resistant depression); migraine and irritable bowel syndrome.

4. A method for combatting a disorder of the type including depressive disorders (but excluding postnatal depression and treatment-resistant depression), migraine and irritable bowel syndrome in the human or non-human animal body, said method comprising administering to said body, a quantity of oestrogen sufficient to increase the amount of SERT.

5. A method of combatting disorders such as migraine, irritable bowel syndrome or depressive disorders (but excluding postnatal depression and treatment-resistant depression) in human or non-human animal body, said method comprising treating the individual with an agent able to cause an increase in SERT mRNA, the amount or activity of SERT.

6. A method of selecting agents able to act as anti-depressants, comprising selecting agents which mimic or affect the association between SERT and oestrogen, or wherein said agents increase the amount of SERT mRNA, of SERT or of the activity of SERT.
